(19) United States
(12) Patent Application Publication
(10) Pub. No.: US 2025/0072990 A1
(43) Pub. Date: Mar. 6, 2025

(54) SEALING DEVICE FOR A SURGICAL INSTRUMENT AND METHOD

(71) Applicant: KARL STORZ SE & Co. KG, Tuttlingen (DE)

(72) Inventors: Michael EGLE, Tuttlingen (DE); Rainer HERMLE, Tuttlingen (DE); Gunther KASPARIK, Tuttlingen (DE); Oliver LÖFFLER, Tuttlingen (DE); Uwe WITTKE, Tuttlingen (DE)

(21) Appl. No.: 18/824,009

(22) Filed: Sep. 4, 2024

(30) Foreign Application Priority Data
Sep. 6, 2023 (DE) 10 2023 123 938.4

(51) Int. Cl. A61B 90/00

(52) U.S. Cl. CPC A61B 90/08

(57) ABSTRACT

A sealing device for at least one surgical instrument, with a sealing element which is designed to seal an interface between a working element and at least one instrument, with a locking element which can be connected to the working element or is formed on the latter. The locking element is designed to fix the sealing element and has at least one latching portion. The sealing element is designed to engage in the latching portion and to be held automatically on the locking element in a position sealing the interface. A method is provided for assembling the sealing device.

### CROSS REFERENCE TO RELATED APPLICATIONS

This application claims the benefit of priority under 35 U.S.C. § 119 of German Patent Application 10 2023 123 938.4, filed Sep. 6, 2023, the entire contents of which are incorporated herein by reference.

### TECHNICAL FIELD

The present invention relates to a sealing device for a surgical instrument and a method for assembling a sealing device.

### BACKGROUND

Surgical instruments are used for different applications. For example, they can be used in the area of minimally invasive surgery and have a surgical tool. Surgical instruments can be designed as resectoscopes and have a working element that is designed, for example, to guide the surgical tool, such as an electrode.

Resectoscopes are particularly well known for urological or, for example, gynecological applications. The working element with the electrode can be inserted into the patient's body through the urethra to a surgical site. At one distal end (i.e. remote from the user), for example, the electrode has a cutting loop that can be used to ablate tissue. The ablated tissue can be removed again through a drain channel by means of an irrigation liquid that is guided through an irrigation channel or inflow channel to the surgical site. The inflow and drain channels enable continuous rinsing, which also serves to keep a viewing window clear to ensure a perfect endoscopic view at all times.

Surgical instruments generally have a handle with a slide that is mounted such that it can be moved along a longitudinal axis of the surgical instrument and is connected to the surgical tool. If an instrument, such as an electrode, is inserted into the working element, the cutting loop of the electrode can be moved in the axial direction by moving the carriage axially, for example to ablate tissue.

The area where the electrode is inserted into the working element must be sealed. Various sealing elements are known for this purpose.

For example, there are sealing elements made of PTFE (polytetrafluoroethylene, also known as “Teflon®”). Such Teflon® elements, such as Teflon® bushes, can be used manually for sealing. However, replacing such sealing elements, for example for cleaning purposes, is time-consuming. Another disadvantage is that sealing by such Teflon elements can only be ensured if an instrument is inserted into the working element. If the instrument is not inserted into the working element, this results in an opening that does not seal.

A protective device for a sealing element is also known from publication EP 2 143 393 B1, wherein a lid with lateral tabs can snap onto the instrument shaft. Two opposing tabs with latching protrusions are arranged on the lid. The latching protrusions can snap into corresponding latching edges on the sealing element holder on the instrument.

Disadvantageously, the sealing element cannot be removed as a complete element through the hook mounting, as the mounting only mounts the lid of the seal. This means that the cost of replacing the complete sealing unit remains high.

Due to the multiple use of known sealing elements with cleaning processes between applications, the sealing elements are generally of a complex design.

### SUMMARY

Against this background, the present invention is based on the object of providing an improved sealing device or method for assembling a sealing device.

According to the invention, this object is achieved by a sealing apparatus with the features of the present invention and/or by a method with the features of the present invention.

Accordingly, the following are provided:

A sealing device for at least one surgical instrument, with a sealing element which is designed to seal an interface between a working element and at least one instrument, with a locking element which can be connected to the working element or is formed on the latter, wherein the locking element is designed to fix the sealing element and has at least one latching portion, wherein the sealing element is designed to engage in the latching portion and to be held automatically on the locking element in a position sealing the interface.

A method of assembling a sealing device for sealing an interface between a working element and at least one instrument of a surgical instrument, comprising the steps of: positioning a sealing element at an angle to a locking element, wherein the locking element is connected to or formed on the working element, engaging the sealing element in at least one latching portion of the locking element, pivoting the sealing element in a plane parallel to the locking element until a sealing position is reached, and holding the sealing element in the sealing position.

The insight on which the present invention is based is that multiple use is not always practical if the sealing elements have to be replaced quickly and easily, for example when the instruments are replaced. By implementing a simple design, on the other hand, a “single use” seal designed for single use can be created.

The idea underlying the present invention is to engage the sealing element on a locking element, wherein the sealing element can be removed from or fixed to the locking element by simple hand movements.

The sealing device can extend at least partially circumferentially around a part of the working element, wherein the part can be aligned in the direction of the handle.

The at least one instrument can be an electrode, needle, laser fiber or similar.

One recess per instrument can be provided in the sealing device. Two separate recesses can also be provided in the sealing device for each instrument. Sealing can therefore take place at least between the respective recess and the respective instrument.

In addition, such a sealing device can also be used to create a seal if no instrument has yet been inserted or pushed into the working element. This enables improved sealing in different conditions, especially with or without instruments inserted.

In this manner, it is also advantageously possible to design the sealing element for single use or as a “single use” sealing element and to use the locking element several times.

Furthermore, the sealing element can be used to ensure that the instrument can be moved smoothly in the seal. For this purpose, the sealing device may have a soft component, in particular the sealing element.

In particular, the sealing element can be surface-treated, for example by fluorination, so that the at least one instrument can be guided particularly smoothly and easily. For example, only one instrument can be introduced. Furthermore, two, three or more than three instruments can be inserted separately, wherein each of the instruments can be guided in its own recess.

The locking element can have different embodiments, for example it can be formed in or on the working element. In particular, this means that the latching portion is formed directly in or on the working element. Different sealing elements can therefore be easily and quickly engaged or removed from the latching portion.

A latching portion is, for example, a type of engagement in which the sealing element can engage. The latching portion can be an undercut. In particular, the latching portion can be formed from more than one engagement or more than one undercut.

In particular, the latching portion can accommodate an edge area of the sealing element in order to hold the sealing element automatically on the locking element.

In particular, automatic means that the sealing element can be held on the locking element without further fixing elements and/or handles. This can be done by engaging and/or clamping in the latching portion, especially in a one-handed operation.

The surgical instrument may be a resectoscope. Use with other surgical instruments is also conceivable.

Advantageous embodiments and developments are shown in the present invention and in the description with reference to the figures in the drawing.

According to an advantageous embodiment, the sealing element can be arranged on a base plate, wherein the base plate has a latching portion corresponding to the latching portion. Advantageously, the sealing device thus comprises a hard component and a soft component. The base plate can be designed as a hard component. Accordingly, the sealing element is designed as a soft component. The latching portion on the base plate can, for example, be designed as a lug that can engage and/or snap into a depression or undercut on the locking element.

The base plate can be larger than the sealing element such that the latching portion protrudes from one side edge of the sealing element, for example. Furthermore, the base plate can also project beyond the sealing element on a side opposite the latching portion, resulting in a further fixing option on a surface of the base plate.

According to one embodiment, the base plate may have a fixing portion, in particular a protrusion, which in the sealing position engages in a recess in the locking element in order to fix the sealing element in the sealing position on the locking element as determined by the latching portions. The fixing portion can be formed in an area in which the base plate protrudes over the sealing element. This means that the fixing portion does not affect the sealing element. In particular, the fixing portion is formed at a distance from the sealing element and is not in contact with the sealing element.

In a further embodiment, different sealing elements can be assembled on one and the same base plate. This makes it easy to produce identical base plates on which different sealing elements can be arranged.

According to an advantageous embodiment, the latching portion may be formed in a plane with the base plate and may not exceed a thickness of the base plate. This allows the base plate with the sealing element to engage very close to the locking element, thereby sealing the area between the working element and the at least one instrument.

According to a preferred embodiment, the fixing portion can be formed laterally on the base plate, in particular in the shape of a hook, and protrude beyond a thickness of the base plate. The fixing portion can be a protrusion or a pin-shaped element that has a bead-like thickening at one free end. This can, for example, enable the locking element to engage.

According to a particularly preferred embodiment, the latching portion and the fixing portion can be oriented in different directions, wherein the latching portion serves as a stop in a first direction and the recess serves as a receptacle and locking device in a second direction. This allows the sealing element to be pressed against the locking element in a particularly advantageous manner. Advantageously, the latching portion in the locking element and the recess in the locking element are arranged in such a manner that the sealing element is held in the sealing position on the locking element in the engaged state. The fixing portion and the sealing element are therefore preferably arranged on the same surface of the base plate.

According to an advantageous embodiment, the base plate can have two latching portions that protrude from one side edge of the base plate. This is particularly advantageous for securing the sealing element against twisting. Furthermore, the latching portions can be arranged in an area above the at least one instrument on the working element, wherein a larger area can be utilized for fixing the sealing element.

According to a further embodiment, the fixing portion can be arranged centrally between the latching portions. In particular, the fixing portion is not arranged in line with the latching portions, but is offset parallel to them. For example, the latching portions can be arranged in an edge area (side edge) on the base plate. The fixing portion can be arranged on an opposite edge area of the base plate. The sealing element can be arranged in between, wherein it can neither be in contact with the latching portions nor with the fixing portion.

According to one embodiment, the sealing element can be designed as a flat seal and have a substantially flat shape. This means that very little installation space is required for the entire sealing device. Advantageously, the sealing element is arranged as a flat seal parallel to a base plate, wherein the flat seal has a small thickness with the base plate.

In an advantageous embodiment, the sealing element can seal the interface in the sealing position even when the instrument is not inserted and/or the sealing element can be perforated when the instrument is inserted. The sealing element can be pierced with at least one instrument, so that an opening is created for each instrument that is precisely adapted to the diameter of each instrument.

In an advantageous embodiment, the base plate may have at least one recess for the receptacle of the at least one instrument, wherein the sealing element spans the at least one recess. The perforation when inserting each instrument is advantageously located directly within the area in which the sealing element spans the at least one recess. This means that each instrument can also be guided through a respective recess. This means that several instruments can be used side by side, wherein each of the instruments can be sealed separately.

In an advantageous embodiment, the locking element may have at least one edge arranged around an opening for receptacle of the at least one instrument. The edge can be in the form of a bead or a bulge, for example. Several edges can also be provided, each of which is designed as a circumferential bulge around a respective through hole for the receptacle of at least one instrument. The sealing effect with the locking element can be optimized by at least one edge. The edge can be aligned in the direction of the sealing element. The edges can therefore be pressed against the sealing element when the sealing element engages with the locking element. The sealing element can have recesses corresponding to the openings to accommodate the instruments.

According to an advantageous embodiment of the method, the sealing element can therefore seal the interface even when the instrument is not inserted and/or the sealing element can be perforated by inserting at least one instrument.

According to an advantageous embodiment of the method, the sealing element can engage in the locking element with a latching portion corresponding to the latching portion, and the latching portions form an axis of rotation about which the sealing element is rotated from the angled position to the sealing position. In this manner, the sealing element can be arranged particularly close to the locking element, wherein a flat seal is advantageously used.

According to an advantageous embodiment of the method, when the sealing position is reached, the sealing element can be fixed to the locking element with a fixing portion, wherein the fixing portion is designed as a protrusion which engages in a recess in the locking element in the sealing position. The latching portion and the recess in the locking element are arranged at such a distance from one another that the sealing element, in particular with the base plate, is held against the locking portion as soon as the sealing element is in the sealing position. Starting from the angled position, the sealing element therefore only engages on the locking element when it has reached the sealing position.

The above embodiments and developments can be combined with each other as desired, if appropriate. Further possible embodiments, developments and implementations of the invention also include combinations, that are not explicitly mentioned, of features of the invention described above or below with respect to the exemplary embodiments. In particular, a person skilled in the art will also add individual aspects as improvements or additions to the particular basic form of the present invention.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is an isometric view of a surgical instrument with a sealing device;

FIG. 2 is a view of a sealing device on a surgical instrument during assembly;

FIG. 3A is a view of the sealing device;

FIG. 3B is another view of the sealing device;

FIG. 3C is yet another view of the sealing device;

FIG. 4 is a further isometric view of a surgical instrument with the sealing device;

FIG. 5 is a view of a locking element in a possible embodiment.

### DESCRIPTION OF PREFERRED EMBODIMENTS

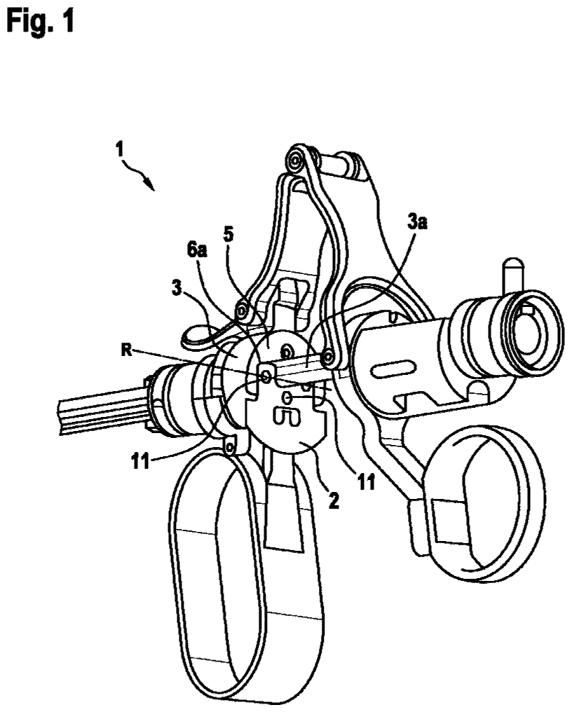

Referring to the drawings, FIG. 1 shows an isometric view of a surgical instrument with a sealing device 1.

A sealing element 2 is designed to seal an interface between a working element 3 and at least one instrument 4 (not shown). A locking element 5 is provided for this purpose, which can be connected to the working element 3 or is formed on it. The locking element 5 can, for example, be snapped, glued or otherwise mounted on the working element 3. The locking element 5 can also be formed in one piece with the working element 3.

The locking element 5 is used to fix the sealing element 2 and has at least one latching portion 6a. The latching portion 6a is arranged on an axis of rotation R about which the sealing element 2 can rotate. This allows the sealing element 2 to be assembled and disassembled quickly and easily. The sealing element can be inserted into the latching portion 6a from below, so to speak, and thus be assembled on the working element 3. In particular, the sealing device 1, or the sealing element 2, is designed for single use, i.e. as a single use seal, and is used, for example, in a resectoscope or for medical instruments in general.

In the illustration in FIG. 1, the surgical instrument is designed for the receptacle of two instruments 4, wherein the sealing element 2 has three recesses 11 (two are visible, one is concealed). The recesses can only be created in the sealing element 2 when the respective instrument 4 is pierced, so that a seal is created when the instrument 4 is not inserted. The recesses 11 are advantageously already present in the base plate 7. At least one marking or perforation can therefore be provided on the sealing element 5 in order to mark the position of the recess 11 in the base plate 7 such that the instrument 4 can be inserted at the corresponding position and the sealing element 5 can be pierced.

The recesses 11 can be arranged at least partially circumferentially around a part 3a of the working element 3 on the sealing device 1.

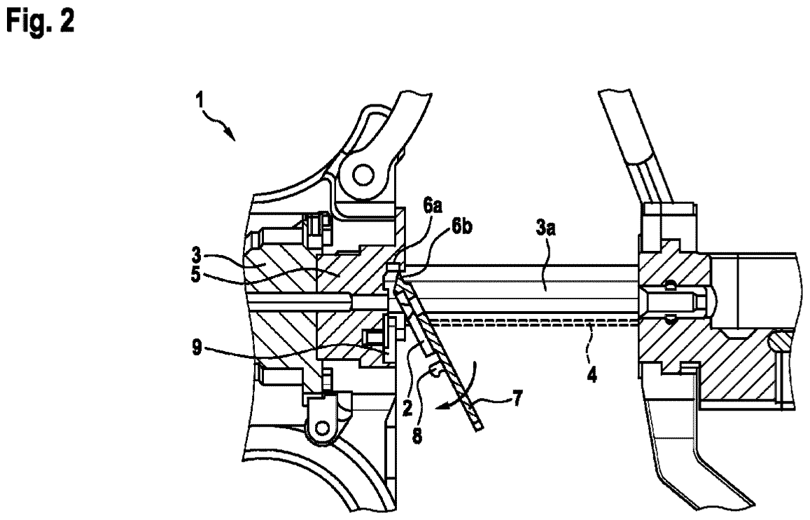

FIG. 2 shows a sealing device 1 on a surgical instrument during assembly.

For assembly or the assembly process, the sealing element 2 is first positioned at an angle to the locking element 5, as shown in FIG. 2. The sealing element 2 is latched into at least one latching portion 6a of the locking element 5, wherein the sealing element 2 in this embodiment has a base plate 7 with a latching portion 6b. The sealing element 2 is then pivoted in such a manner that it is arranged in a plane parallel to the locking element 5 and reaches a sealing position in this position. Parallel means in particular that the sealing element 2 is in full-surface contact with a surface of the locking element 5. For fixing, a fixing portion 8 is provided on the sealing element 2, or on the base plate 7, which protrudes from a surface of the base plate 7 and thus projects beyond the thickness of the base plate 7. The fixing portion 8 and the at least one latching portion 6b are aligned in different directions so that the sealing element 2 can be securely fixed to the locking element 5. The fixing portion 8 can, for example, be hook-shaped or nose-shaped, wherein a bead-like thickening can be provided at a free end of the fixing portion 8. This bead-like thickening can form the hook shape, for example. This allows the fixing portion 8 to engage in a recess 9 in the locking element 5 and fix the sealing element 2 in the sealed position.

An instrument 4 is indicated by dashed lines. This can then be pushed in, allowing the sealing element 2 to be perforated.

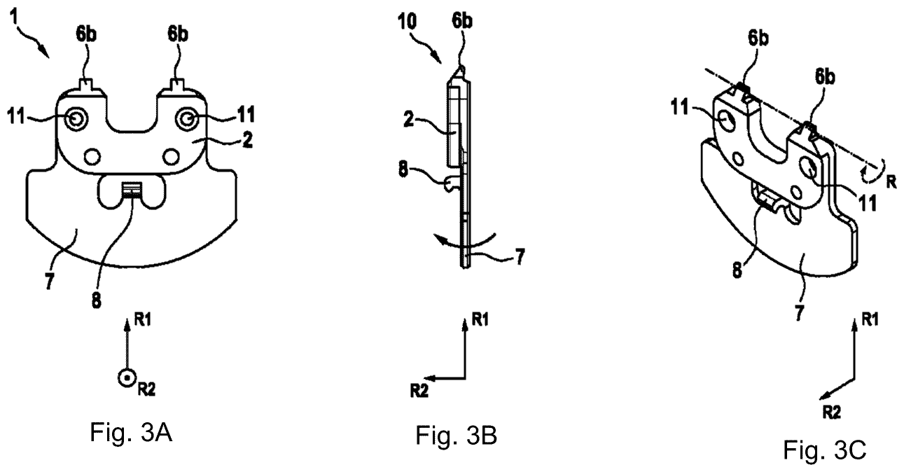

FIG. 3A, FIG. 3B and FIG. 3C are three views of a sealing device 1.

The base plate 7 has two latching portions 6b that protrude from one side edge 10 of the base plate 7. Several recesses 11 can be provided below the latching portions 6b, through each of which an instrument 4 (not shown) can be passed. When the at least one instrument 4 is passed through, the sealing element 2, which spans the recess 11, can be pierced so that an optimum seal can be achieved around each instrument 4. Each instrument can therefore be sealed separately.

For reasons of clarity, the illustration already shows two recesses 11, wherein these can only be indicated or cannot be recognized in the non-punctured state. The arrangement of the recesses 11 may differ from the positions shown.

Advantageously, the sealing device 1 can also have a sealing function before the at least one instrument 4 is inserted. Smooth running of the instrument 4 can be achieved, for example, by treating the surface of the sealing element 2. The sealing element 2 is preferably designed as a soft component, wherein a surface treatment can be carried out, for example, by floring. The base plate 7 is preferably shaped as a hard component and is larger than the sealing element 2. Accordingly, the base plate 7 protrudes above the sealing element 2. The fixing portion 8 can be arranged on the base plate 7 on an opposite side to the side edge 10. The fixing portion 8 is preferably aligned in the spatial directions R2, while the latching portions 6b are aligned in the spatial directions R1. The spatial directions R1, R2 are preferably aligned transversely to one another, in particular orthogonally. This allows the sealing element 2 to be fixed to the locking element 5 in two different spatial directions. During assembly, rotation around the axis of rotation R takes place until the fixing portion 8 is engaged on the locking element 5. When the fixing portion 8 is engaged in the locking element 5, the sealing element 2 is preferably in contact with the entire surface of the locking element 5 and thus aligned parallel to the locking element 5. In particular, in this state the sealing element 2 is pressed against the locking element 5, namely by being fixed by the latching portions 6a, 6b and by the fixing portion 8.

In other words, the sealing element 2 can be fixed to the locking element 5 by a holding element, in the form of the at least one latching portion 6a, 6b, and by at least one snap hook, in the form of the fixing portion 8. This ensures positioning and fixation.

In the embodiment shown, the sealing element 2 is designed as a flat seal such that it requires little installation space. It can also be easily assembled and disassembled, and held securely in position between the working element 3 and the instrument 4 by the different latching or fixing portions.

The fixing portion 8 is preferably provided on the surface of the base plate 7 on which the sealing element 2 is also arranged. This also saves installation space and the sealing element 2 can be securely and tightly fixed to the locking element 5.

Furthermore, the instrument can run smoothly, as each opening in the sealing element 2 can be precisely adapted to the size of the instrument 4. Due to its simple design and few components, the sealing device 1 is suitable for single use and can therefore be implemented economically.

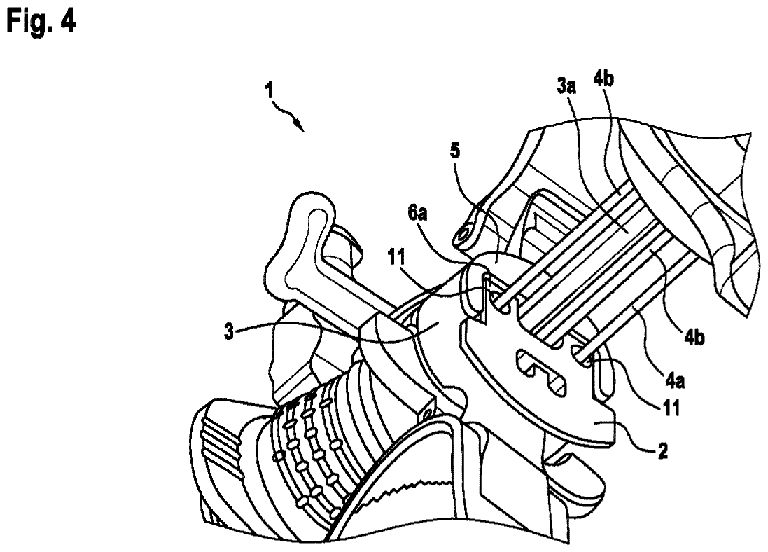

FIG. 4 shows a further isometric view of a surgical instrument with a sealing device 1. Two instruments 4a, 4b can be recognized here. Each of the instruments 4a, 4b can be electrodes, needles, laser fibers or similar. These can be arranged around a part 3a of the working element 3.

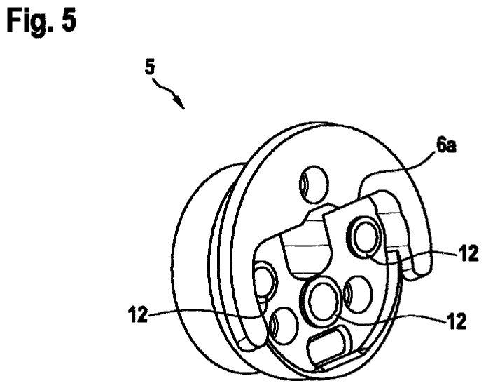

FIG. 5 shows a locking element 5 in a possible embodiment. This can have at least one edge 12 or several edges 12, for example in the form of beads, in order to optimize the sealing effect with the locking element 5. The edges 12 can be aligned in the direction of the sealing element 2. The sealing element 5 has openings corresponding to the recesses 11 to accommodate the instruments 4.

Although the present invention has been fully described above with reference to preferred exemplary embodiments, it is not limited thereto, but can be modified in a variety of ways.

For example, the position of the latching portions 6b or the latching portions 6a may have different embodiments.

Furthermore, the fixing portion 8 can be provided at a different position on the base plate 7.

Likewise, two fixing portions 8 can be arranged on the base plate 7, in particular on a surface of the base plate 7, in order to adapt the sealing device 1 for different assembly situations.

While specific embodiments of the invention have been shown and described in detail to illustrate the application of the principles of the invention, it will be understood that the invention may be embodied otherwise without departing from such principles.

### LIST OF REFERENCE SIGNS

1 scaling device
2 scaling element
3 working element
3a part of the working element
4 instrument
5 locking element
6a latching portion
6b latching portion
7 base plate
8 fixing portion
9 recess
10 side edge
11 recess
12 edge

What is claimed is:

1. A sealing device for at least one surgical instrument, the comprising:
a sealing element configured to seal an interface between a working element and at least one instrument;
a locking element configured to be connected to the working element or is formed on the working element, wherein the locking element is configured to fix the sealing element and the locking element has at least one latching portion, wherein the sealing element is configured to engage in the latching portion and to be held automatically on the locking element in a position sealing the interface.

2. The sealing device according to claim 1, wherein the sealing element is arranged on a base plate, wherein the base plate has a latching portion corresponding to the at least one latching portion of the locking element.

3. The sealing device according to claim 2, wherein the base plate has a fixing portion comprising a protrusion, which in the sealing position engages in a recess in the locking element to fix the sealing element in the sealing position on the locking element as determined by the at least one latching portion of the locking element and the latching portion of the base plate.

4. The sealing device according to claim 2, wherein the latching portion of the base plate is formed in a plane with the base plate and the latching portion of the base plate does not protrude beyond a thickness of the base plate.

5. The sealing device according to claim 3, wherein the fixing portion is formed laterally on the base plate in a shape of a hook, and the fixing portion projects beyond a thickness of the base plate.

6. The sealing device according to claim 3, wherein the latching portion of the base plate and the fixing portion are oriented in different directions, wherein the at least one latching portion of the locking element serves as a stop in a first direction and the recess serves as a receptacle and locking in a second direction.

7. The sealing device according to claim 2, wherein the base plate has another latching portion to provide two latching portions projecting from a side edge of the base plate.

8. The sealing device according to claim 7, wherein the fixing portion is arranged centrally between the latching portion of the base plate and the another latching portion of the base plate.

9. The sealing device according to claim 1, wherein the sealing element is configured as a flat seal and the sealing element has a substantially flat shape.

10. The sealing device according to claim 1, wherein the sealing element seals the interface in the sealing position when the at least one instrument is not inserted and/or the sealing element undergoes at least one perforation as a result of insertion of the at least one instrument.

11. The sealing device according to claim 2, wherein the base plate has at least one recess for receiving the at least one instrument, wherein the sealing element spans the at least one recess.

12. The sealing device according to claim 1, wherein the locking element has at least one edge arranged around an opening for receiving the at least one instrument.

13. A method for assembling a sealing device for sealing an interface between a working element and at least one instrument of a surgical instrument, with a sealing device according to claim 1, the method comprising the steps of:
positioning a sealing element at an angle to a locking element, wherein the locking element is connected to the working element or is formed on the working element;
engaging the sealing element in at least one latching portion of the locking element;
pivoting the sealing element in a plane parallel to the locking element until a sealing position is reached; and
holding the sealing element in the sealing position.

14. The method for assembling according to claim 13, wherein the sealing element seals the interface when the at least one instrument is not inserted and/or the sealing element undergoes at least one perforation when the at least one instrument is inserted.

15. The method for assembling according to claim 13, wherein the sealing element engages in the locking element with a latching portion corresponding to the latching portion of the locking element, and the latching portion of the locking element and the latching portion of the sealing element form an axis of rotation about which the sealing element is rotated from an angled position to the sealing position.

16. The method for assembling according to claim 13, wherein, when the sealing position is reached, the sealing element is fixed to the locking element by a fixing portion, wherein the fixing portion is configured as a protrusion which engages in a recess in the locking element in the sealing position.

17. A method for assembling a sealing device for sealing an interface between a working element and at least one instrument of a surgical instrument, the method comprising the steps of:
providing a sealing device comprising a sealing element and a locking element connected to the working element or formed on the working element;
positioning the sealing element at an angle relative to the locking element, wherein the sealing element engages at least one latching portion of the locking element; and
pivoting the sealing element in a plane parallel to the locking element until a sealing position is reached, wherein the sealing element is held in the sealing position.

18. The method for assembling according to claim 17, wherein the sealing element seals the interface when the at least one instrument is not inserted and/or the sealing element undergoes at least one perforation when the at least one instrument is inserted.

19. The method for assembling according to claim 17, wherein the sealing element engages in the locking element with a latching portion corresponding to the latching portion of the locking element, and the latching portion of the locking element and the latching portion of the sealing element form an axis of rotation about which the sealing element is rotated from an angled position to the sealing position.

20. The method for assembling according to claim 17, wherein, when the sealing position is reached, the sealing element is fixed to the locking element by a fixing portion, wherein the fixing portion is configured as a protrusion which engages in a recess in the locking element in the sealing position.
